(19) United States
(12) Patent Application Publication
(10) Pub. No.: US 2019/0269395 A1
(43) Pub. Date: Sep. 5, 2019

(54) FAR CORTEX INTRAOSSEOUS SUTURE ANCHOR

(71) Applicant: Syntorr, Inc., Palo Alto, CA (US)

(72) Inventor: Daniel L. MARTIN, Palo Alto, CA (US)

(21) Appl. No.: 16/291,688

(22) Filed: Mar. 4, 2019

Related U.S. Application Data
(60) Provisional application No. 62/637,838, filed on Mar. 2, 2018.

(51) Int. Cl. A61B 17/04

(52) U.S. Cl. CPC A61B 17/0401; A61B 2017/0414; A61B 2017/0451; A61B 2017/0459

(57) ABSTRACT

A suture anchor includes a bone attachment portion configured for engagement in cortical bone, and a body portion that is coupled to the bone attachment portion and includes a plurality of holes for passage of sutures.

### CROSS-REFERENCE TO RELATED APPLICATION(S)

This application is based upon and claims the benefit of priority from U.S. Provisional Patent Application Ser. No. 62/637,838, filed Mar. 2, 2018, the entire contents of which are incorporated herein by reference.

### BACKGROUND

The present disclosure generally relates to devices and methods for repairing bone fractures, and more specifically to improved far cortex intraosseous suture anchor.

### SUMMARY

A suture anchor, according to an embodiment, includes a bone attachment portion configured for engagement in cortical bone, and a body portion that is coupled to the bone attachment portion and includes a plurality of holes for passage of sutures.

In one embodiment, the body portion has a length that is at least one-fourth of an entire length of the suture anchor. In another embodiment, the bone attachment portion has a length that is less than one-fourth of an entire length of the suture anchor.

In some embodiments, each of the bone attachment portion and the body portion has a generally cylindrical shape, wherein a diameter of the bone attachment portion is less than a diameter of the body portion.

The suture anchor according to an embodiment further comprises a suture locking mechanism. The suture locking mechanism is configured to secure the sutures passing through the holes to the body portion. The suture locking mechanism includes a movable wedge that is pressed against the body portion to secure one of the sutures passing through one of the holes to the body portion.

In one embodiment, the movable wedge has a plurality of tines formed on a surface thereof that presses the suture against the body portion. The tines are spaced from each other at an interval that is less than the diameter of the suture and are tilted in a first direction that is opposite a second direction in which the movable wedge is moved to press the suture against the body portion.

In another embodiment, the suture locking mechanism includes multiple wedges that are mechanically linked to move together to press the sutures passing through the holes against the body portion to secure the sutures to the body portion.

The suture anchor according to another embodiment further comprises a suture locking mechanism that includes a surface locking mechanism. The surface locking mechanism is attached to the sutures passing through the holes and pressed against the body portion.

### BRIEF DESCRIPTION OF THE DRAWINGS

So that the manner in which the above recited features of the present disclosure can be understood in detail, a more particular description of the disclosure, briefly summarized above, may be had by reference to examples, some of which are illustrated in the appended drawings. It is to be noted, however, that the appended drawings illustrate only exemplary examples and are therefore not to be considered limiting of its scope, may admit to other equally effective implementations.

FIG. 1 shows a cross section of a proximal humerus to which a suture anchor according to an embodiment is attached.

FIG. 2 shows a diagrammatic suture anchor with multiple holes along the length of the body of the suture anchor.

FIG. 3 shows a different configuration of the suture anchor of FIG. 2 in which the holes in the body of the suture anchor may be positioned side-by-side.

FIGS. 4A-4C illustrate different means for restraint of the suture in the suture anchor.

FIGS. 5A-5D illustrated various suture locking mechanisms.

FIGS. 6A-6C show a locking mechanism with mechanical tines that engage a suture, according to various embodiments.

FIGS. 7A-7F show various bone attachment mechanisms, according to various embodiments.

FIG. 8 shows the driver portion of the suture anchor.

FIG. 9 shows the relationship in length between the BAM, body, and driver, according to various embodiments.

FIG. 10 shows an instrument for use in surgical application of the anchor, according to various embodiments.

FIG. 11 shows a drill guide, according to various embodiments.

FIG. 12 shows a passing loop that may be positioned in the holes or suture channels of the anchor, according to an embodiment of the present disclosure.

FIGS. 13A-13F show the suture anchor according to various embodiments.

FIG. 14 shows an insertion tool, according to an embodiment.

FIG. 15A shows a conventional suture anchor in comparison to the suture anchor according to an embodiment.

FIGS. 15B and 15C demonstrate an advantage of the suture anchor according to an embodiment.

To facilitate understanding, identical reference numerals have been used, where possible, to designate identical elements that are common to the figures. It is contemplated that elements and features of one implementation may be beneficially incorporated in other implementations without further recitation.

### DETAILED DESCRIPTION

### Definition of Terms

Suture: Refers to one or more surgical sutures as used for repairing tissues in surgery.

Tether: This is a string or cord, may be a suture material, but is not used for repairing tissues and surgery. For example, in some embodiments this is the passing loop. It may be fabricated from suture but it is a fundamentally different element in that it is not used for repairing tissue.

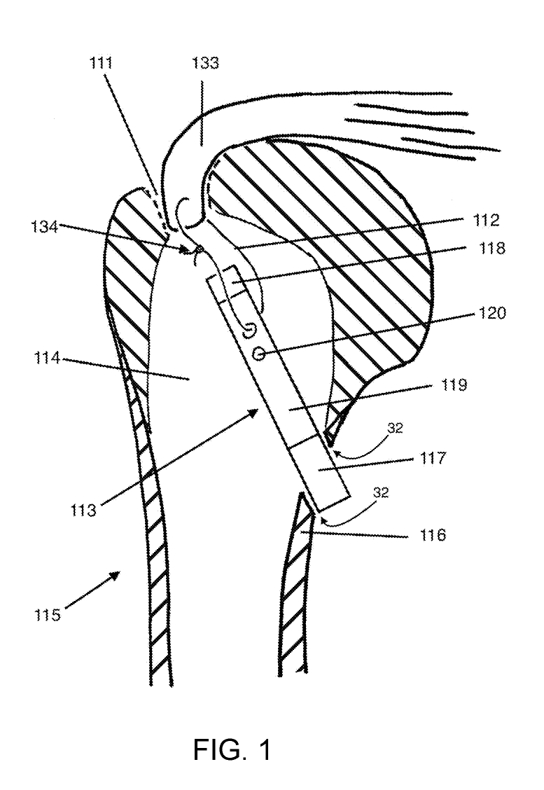

Far Cortex Anchorage (FCA): FCA refers to a technique of attachment of a suture anchor to a far cortex of bone, as discussed below. It is discussed in U.S. Pat. No. 9,289,202, and the novelty of the FCA method has been defined. Some existing conventional anchors can be made to attach to the far cortex, using the technique of FCA. However, conventional suture anchors for rotator cuff repair are designed to anchor in the relatively weak thin cortical bone and shallow cancellous bone located in the area of the recess 111 shown in FIG. 1. Prior art conventional anchors are designed adapting to the constraints of shallow weak cancellous bone, according to usual conventional anchor application. In contrast, the suture anchor construct (SAC) described herein is a suture anchor system that is designed specifically for attachment to the far cortex 116, designed specifically for the FCA technique, and designed so that it is not optimized for application in the conventional anatomic location for placement of rotator cuff suture anchors. As such, SAC accommodates the different anatomic, spatial, size, suture capacity, and other needs of the FCA technique, and optimizes the benefits that can be achieved through using the FCA technique.

The SAC is a suture anchor designed for a novel application, and as such may be classified as a special kind of anchor. It is a type of an FCA anchor. There are other suture anchors designed specifically optimized for the intended anatomic greater tuberosity placement location, such as the corkscrew anchors, and as such have specific attributes. Yet, other anchors are adapted specifically to accommodate dense cancellous bone, such as in the glenoid margin, or acetabular margin. The SAC embodiments of this specification are specifically and uniquely adapted to the anatomy of FCA application. SAC represents a new and separate class of suture anchor.

SAC is a construct for attaching soft tissue elements to bone, using the FCA surgical method. SAC devices and methods support the FCA method. The construct includes at least a suture, a suture anchor, and a means for restraining the suture from movement with respect to the suture anchor. SAC may also include a suture anchor with a body portion, a bone attachment portion, and a driver portion. It may also include a suture locking means, a passing loop, and an insertion tool.

The SAC according to embodiments includes at least a suture anchor with bone attachment means (BAM), a body, a suture, and a means of restraint 134 of the suture. This means of restraint may include a knot or a suture lock. The SAC may additionally include a passing loop that is made from a tether and this passing loop may consist of a continuous passing loop. It may additionally include an insertion tool.

### Introduction

The SAC, according to various embodiments, has a unique application. It is designed for placement within a central cavity of bone, with fixation to the far wall or far cortex of the bone. This technique may be referred to a far cortex anchorage, or FCA. The word “far” derives from the position relative to the surgeon. The near cortex is on the side of the bone that is near to the surgeon, in this case the thin cortex at the attachment tuberosities of the humerus. The near cortex is what the surgeon visualizes as near at the time of surgery. The central cavity of the bone may be defined as a cavity in the bone with substantially fatty marrow, or a zone of low-density cancellous bone. This cavity is more pronounced in the age group of patient that typically present with rotator cuff tears. The fatty space or low density cancellous bone in the central cavity is such that much better mechanical attachment is achieved on a far wall of bone than in the central cavity. FCA differs from the standard design paradigm of suture anchors in that FCA attaches to a far cortex for mechanical strength. The standard conventional suture anchor attaches to a near cortex and to the cancellous bone adjacent to the near cortex. The site of usual suture anchor placement in rotator cuff surgery is in a location where there is only a thin cortical shell, and the underlying cancellous bone is shallow. Going deeper with the anchor only goes into the central cavity, where there is no bone for the anchor to hold onto. Therefore the usual technique is to preserve the thin cortical shell, for strength. Preserving the cortical shell causes a barrier to healing, with delayed failures of the repairs.

In contrast, anchoring in the far cortex allows the surgeon to expose cancellous bone in the area of healing at the tendon attachment site, without weakening attachment. This results in much better biological healing. Furthermore the far cortex is much stronger that at the tendon attachment site, so much so that mechanical failure at the far cortex has never been seen.

The contact area for the bone attachment at the far cortex is small because the bone there is very strong. In contrast, the anchor designers have tried to maximize the hold of anchors by increasing the profile of the conventional anchors, making them bigger, with bigger threads. This is the opposite of the design requirement for an anchor in the dense bone of the far cortex, where the bone engagement can be small.

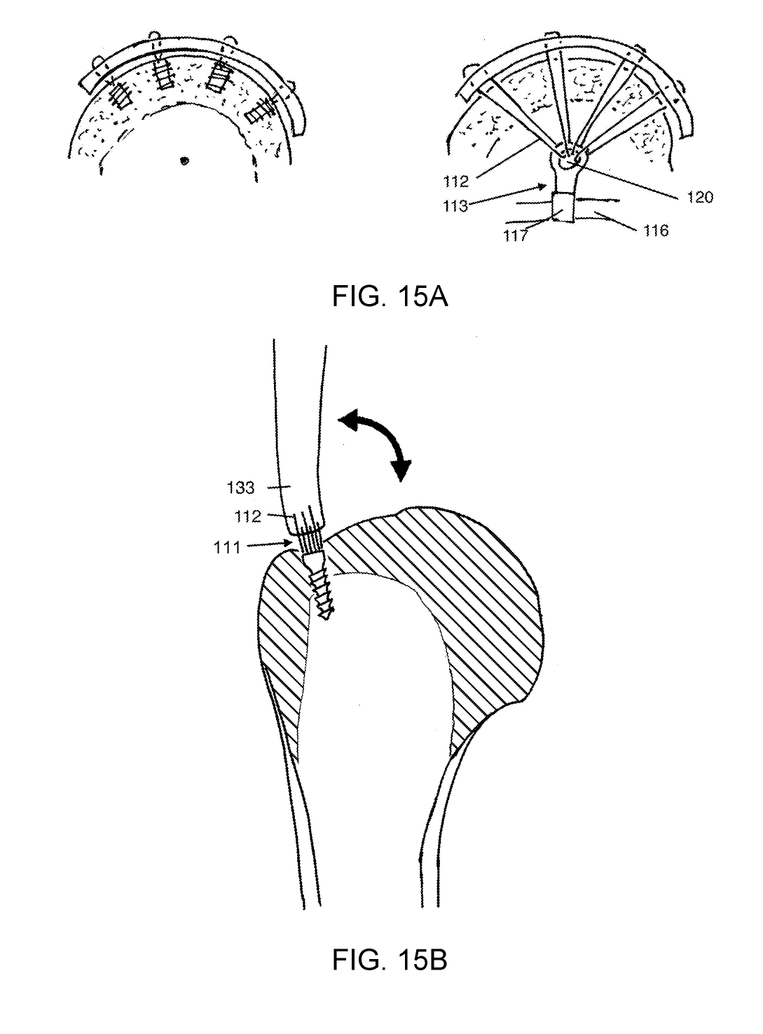

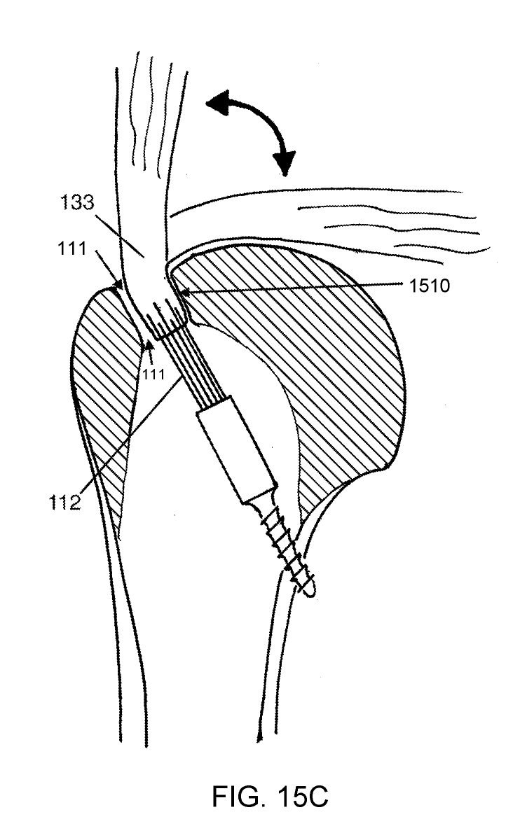

In FIG. 15A, the comparison to conventional suture anchors in conventional use is illustrated. The view in FIG. 15A is substantially orthogonal to the view in FIG. 1. FIG. 15A shows conventional anchors on the left, holding to the bone adjacent to the tendon attachment site. In order to have distributed points of suture loading on the tendon, multiple anchors are used, and there is no distance between the anchors and the tendon. In contrast, with FCA and SAC, there is plenty of space and between the points of suture hold on anchor and suture hold on the tendon. With a conventional anchors in conventional use, the anchor is placed in the bone immediately under the tendon that it is repairing, providing no distance for the sutures to converge from spaced attachment sites on the tendon to a single common attachment site on the anchor. As a result, the common convention with conventional suture anchors is to use multiple anchors and spread them apart on the bone, so that the points of fixation on the tendon can be distributed. In contrast, when SAC is applied as described herein for far cortex anchoring, a distance between the edge of the recess 111 and anchor 113 is preserved and is used for clinical benefit. The distance also provides space for suture mass and knotless suture locking mechanisms. With conventional anchors placed near the surface at the tendon attachment site, there is no space or distance between the anchor and the tendon.

As also shown in FIG. 15A, the head of the humerus has a basically spherical upper surface, so that for an anchor located near the center of the head, the direction of pull of the suture to the tendon is substantially in a direction normal to the bone surface. This makes it so that the tendon at each point of suture attachment is pulled in a direction substantially normal to the bony surface. Suture tension on the tendon that is normal to the humeral head surface provides an optimal direction of pull to hold the tendon against the head. For suture attachment to that anchor at the bone surface, tension on distributed locations on the tendon would cause bunching of the tendon. Configuring an anchor adapting to this head geometry and biomechanical fact is part of the inventive process, and is represented in this specification.

This different paradigm for use makes novel certain design elements of the SAC. The central location in the bone makes available much more volume space for the SAC, and certain embodiments take advantage of means that use this volume to clinical benefit. SAC is sized to position suture attachment to the anchor at a location near the center of the humeral head. The central location, in the approximately spherical head structure, of the suture attachment points on the anchor allows the repair sutures to converge at the anchor, which is a distance from the soft tissue the repair sutures are attached to, as shown in FIG. 15A. Through the convergence of the repair sutures in this way, distributed load on the soft tissue being repaired is preserved. This is similar to the weight of a parachutist being distributed evenly around the margins of the parachute by the cords that pass from the parachute margins and converge to the central dangling person. This makes it so that many or all of the sutures of a cuff repair can be attached to one anchor, something that is not possible with commonly used suture anchors. This is a one-anchor rotator cuff repair and cuff repair device.

FIGS. 15B and 15C demonstrate an advantage of the SAC, compared to prior art. FIG. 15B represents a conventional anchor tendon repair. With movement of the shoulder, the tendon lifts away from the surface of the humeral head, causing a gap to form at the intended site of healing of the tendon to the humerus. Movement also occurs at this site. The gap and movement prevent healing, and eventually the suture repair fails, before healing can occur. In comparison, FIG. 15C shows SAC with the FCA technique. The tendon is drawn over the edge 1050 of the recess 111 made to receive the end of the tendon, and drawn down into the recess 111. Due to the change in angle of the tendon as it is drawn over the edge, the end of the tendon does not lift up or move as the shoulder is moved into forward flexion or abduction. Forward flexion is represented by the position of the tendon pointing upward in FIG. 15C, showing it orthogonal to the tendon lying flat against the humeral head. The end of the tendon is to held stable in the recess 111, and scar formation and other healing is allowed to proceed. Furthermore, the recess 111 exposes the tendon to bleeding cancellous bone, bringing healing factors to the healing interface that are not available at the cortical surface of the normal tendon attachment footprint. The SAC is configured to accommodate these mechanical and biological factors that bear on the healing process.

Design elements of the SAC are novel in the context of intended application. For example, the single SAC is designed to accommodate many sutures, because convergence allows many sutures to meet at the SAC. This allows a single anchor to be used instead of multiple anchors. This specification discloses means and design features to adapt to arthroscopic application even though the SAC is substantially larger than prior art suture anchors. The SAC is generally elongate in form, to allow insertion through an arthroscopic portal. Various embodiments include design elements and means and specific devices to make anchoring to the far cortex practical and commercially viable.

Another design optimization for the FCA is having a small diameter BAM.

Typically, the transverse dimension of the BAM does not need to be greater than 4 mm for rotator cuff repair. A larger size bone hole 132 will weaken the bone. Holding power obtained by larger BAM is not necessary, though in certain cases may be desirable. For the body however, there is no liability in having a lager transverse dimension and it may be very beneficial in allowing efficient knotless locking mechanisms. Therefore, for certain embodiments, a body diameter is larger than the BAM diameter.

In summary, the FCA anchor configuration shortens duration of surgery, through use of just one anchor and knotless locking mechanisms that are contained within the central cavity. The anchor configuration also improves the biology of the healing, by drawing the tendon against bleeding cancellous bone, and holding it there without movement during the healing period. The design features of the FCA allow these features to be active, by applying different relative sizes of the bone engagement portions and suture holding portions, and by accommodating a greater number of sutures locking to one anchor, accommodating multiple sutures and bundles of suture up to many holes in the FCA, and by accommodating an insertion technique that works with arthroscopic surgery. Various embodiments unite these features to enable efficient application of the suture anchor to the far cortex.

### DETAILED DESCRIPTION

FIG. 1 shows a cross section of a proximal humerus 115. There is a rotator cuff tendon which is drawn into a recess 111 in the top of the humeral head, by a suture 112, and this tendon is attached to a suture anchor 113 with the suture 112. The suture anchor 113 is substantially larger than most suture anchors that are typically used for rotator cuff repair, because it resides mostly in the central cavity 114 of the humeral head, rather than near the surface of the humeral head near the rotator cuff tendon anatomic attachment. The common and conventional surgical method for rotator cuff repair uses small anchors that are positioned in the area of the trough 111. The end of the suture anchor having bone attachment means (BAM) 117 intersects and engages with the medial humeral cortex 116, engaging strong dense cortical bone in this location, so that the anchor 113 is resistant to being pulled out by traction on the suture 112. Multiple sutures 112, even greater than ten, may be used and are considered to be used between the anchor and the rotator cuff tendon 133. The suture anchor typically has a bone attachment portion 117 that engages the bone, which is shown in engaging the medial cortex 116. It also typically has a driver interface portion 118 that is used to hold the anchor, for the purpose of insertion. A suture anchor also typically has a channel for passage of a suture. For the purpose of this disclosure, the anchor will be considered to have three main components, the driver 118, the body 119, and the BAM 117. To differentiate the embodiments from other prior art suture anchors, the body of this anchor is separate from the bone attachment means. The usual suture anchor has a common body and bone attachment means, in an effort to maximize the engagement surface area between the suture anchor and the cancellous bone. In this case, the suture anchor engages strong cortical bone, and does not rely upon the body 119 for its hold on the bone, and therefore the function of the body 119 is separate and different from that of the BAM 117 at the end.

In FIG. 1, the suture anchor 113 is shown with two holes 120 through the anchor body. These holes represent suture passageways through the anchor. In various embodiments, a single hole or multiple holes through the body are also considered. One hole, two holes, three holes, four holes, five holes, and greater than five holes are contemplated, and this is unique compared to prior art in suture anchors. Prior art suture anchors typically have only one passage hole for repair sutures, and at times this single passage hole is used for multiple sutures.

The SAC, in some embodiments, includes the suture anchor 113 with one or more sutures, and a means for restraint of the suture so that it does not slide out of the hole. In some embodiments, the SAC is the construct including the suture anchor, the suture, and the means for restraint of one or more sutures.

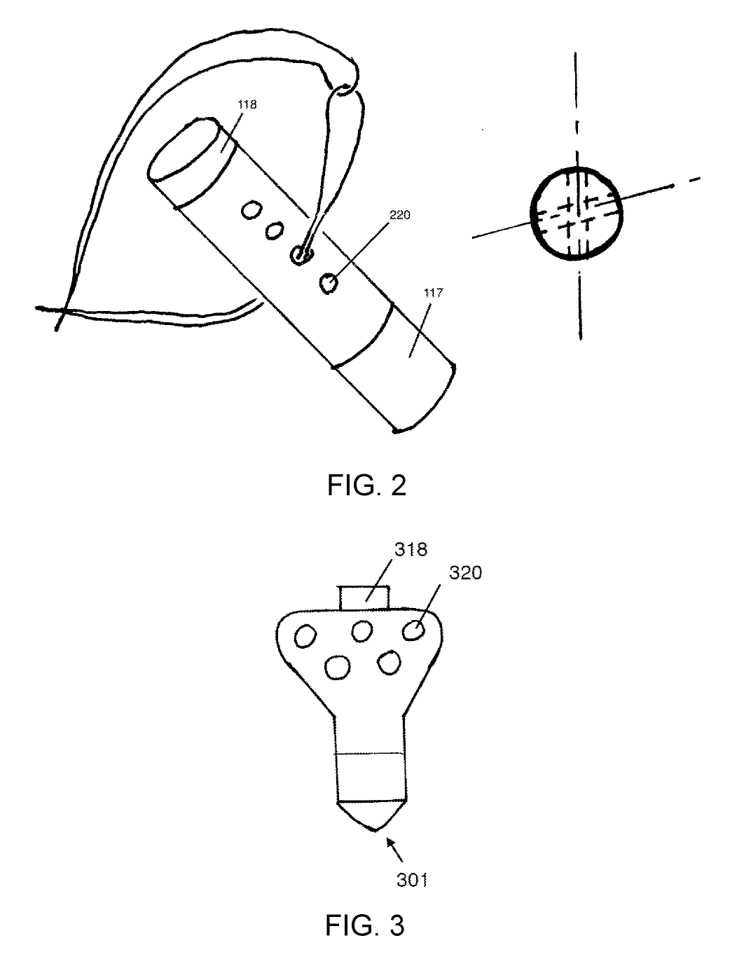

FIG. 2 shows a diagrammatic suture anchor with multiple holes along the length of the body of the suture anchor. The holes 220 are passageways for suture, and they are arranged substantially along the length of the body 219 of the suture anchor. The holes are sized so each hole may pass either one suture or multiple sutures, according to intended use. The holes maybe in a parallel direction or arranged in different radial directions with respect to the axis of the suture. The holes 220 may be aligned at different angles with respect to the axis of the suture anchor 213. The holes may contain knotless locking mechanisms to restrain the sutures.

FIG. 3 shows a different configuration of the suture anchor where the holes in the body of the suture anchor may be positioned side-by-side. in other words, the distance from the tip 301 of the suture anchor, at the bone attachment end, to the suture passage hole 320 may be the same as the distance from the tip 301 of the suture anchor to another hole 320, or the difference in the distances from the tip 301 is less than one diameter of the hole 320.

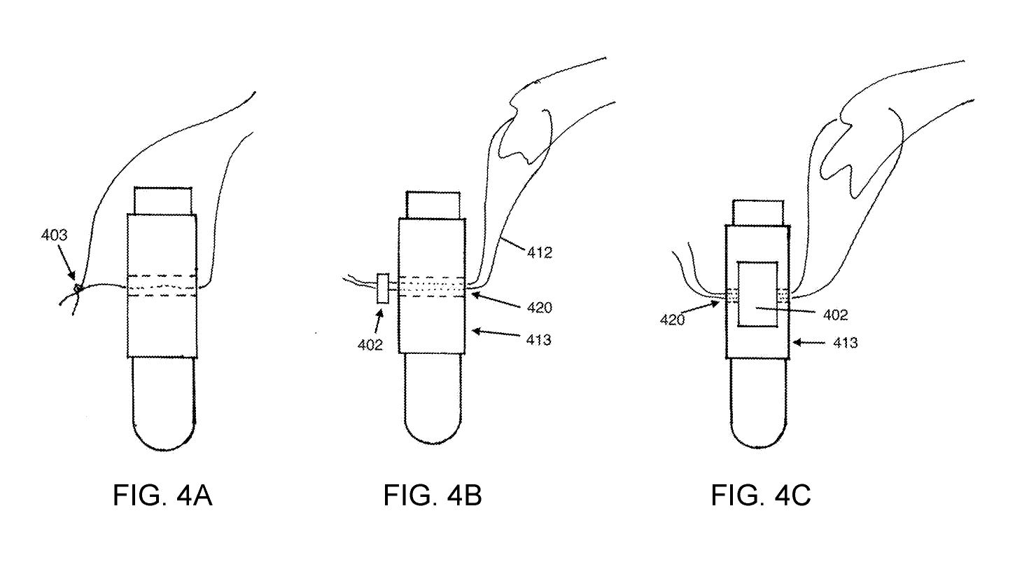

FIGS. 4A-4C address the means for restraint of the suture in the SAC. Another way to say restraint is to say that the suture is locked with respect to the suture anchor to prevent movement at least in one direction. FIG. 4A shows the most basic means of restraint which is a knot 403 in the suture. FIG. 4B shows two suture ends coming through the passage hole 420 in the anchor 413, and a suture locking mechanism is affixed to the suture, such that traction on the suture 412 will cause the suture lock to slide against the entry to the hole, thereby preventing further sliding, providing restraint of movement. This is a suture locking mechanism 402 that is attached only to the suture. There are several examples of suture locks in the commercial and patent literature, providing a speedy way to apply a lock to a suture, and to lock two or more sutures together at the point of the lock.

FIG. 4C shows the SAC including suture, suture anchor 413 and a suture locking mechanism 402 that is integrated within the body of the anchor. In this configuration, the ends of the suture may be drawn through the hole 420 or channel in the body of the suture anchor, and the suture locked without applying a knot or external suture locking mechanism. For the sake of illustration, only one passage hole is shown. In other embodiments, two, three, four, or five independent locking mechanisms may be included in the same suture anchor. This adapts to this unique anatomic situation where an embodiment is configured to allow all sutures from the cuff to be restrained in one suture anchor. Having multiple independent locking mechanisms is beneficial to the situation where many sutures are fixed in one anchor. In certain locking mechanisms there is a maximum number of suture strands that can be locked in in one suture lock, for example six strands. Therefore, greater than six strands from the cuff tendon, to be locked in one anchor, requires more than one hole and locking mechanism in that suture anchor.

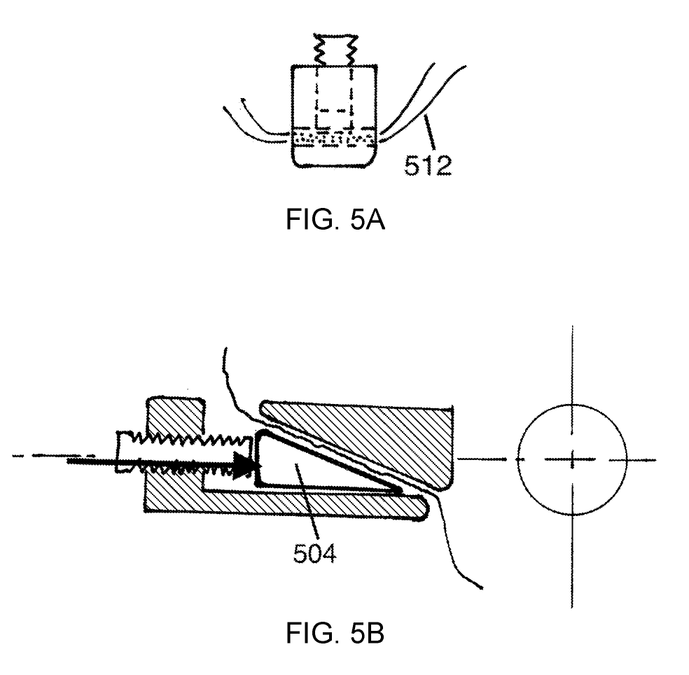

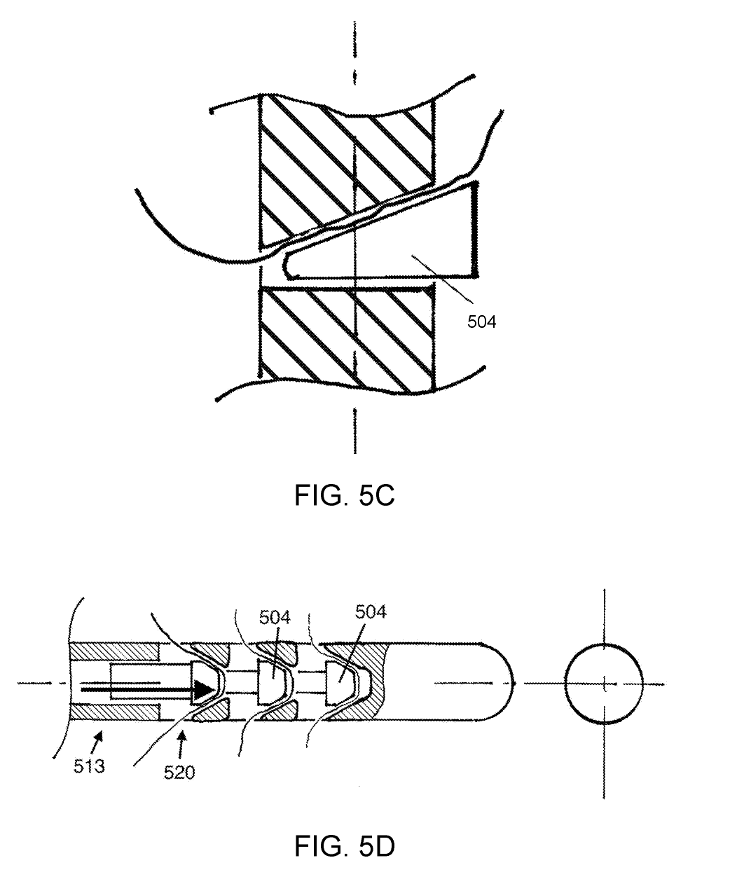

FIGS. 5A-5D illustrate various suture locking mechanisms, beyond that known in the prior art, and means that may be incorporated in embodiments. Prior art suture locks may also be incorporated uniquely into the SAC embodiments. The first is shown in FIG. 5A, where a screw device and plunger is applied directly to the sutures 512 passing through the locking mechanism, applying force, and preventing movement and slipping of the sutures. This in isolation is known in the prior art, but not in combination with the additional features of the SAC as described herein. FIG. 5B shows a wedge device where the suture passes through the lock, and the wedge 504 moves substantially longitudinal with the axis of the locking mechanism or suture anchor. A threaded device is shown applying a sliding force on to the wedge, closing the gap on to the suture, thereby locking the suture. Other means of applying this sliding Force are also contemplated, including traction on the suture from the repaired tendon, and a removable force application mechanism.

FIG. 5C shows a wedge that moves in a direction transverse to the axis of the suture anchor or axis of the independent suture locking mechanism, locking the suture as the traction is applied to the suture causing sliding of the wedge and closure of the gap. Greater force applied to the suture in the direction of the repaired tissue causes a tighter pinch against the suture, the wedge being drawn in the direction of the pull. This is an example of a locking mechanism with a wedge, where the motion of the wedge is in a direction that is substantially transverse to the longitudinal axis of the suture locking mechanism or suture anchor body.

FIG. 5D shows a suture locking mechanism that includes multiple wedges that are mechanically linked to move together, such that multiple passage holes 520 in the suture anchor 513 can be locked with movement of the linked group of wedges 504.

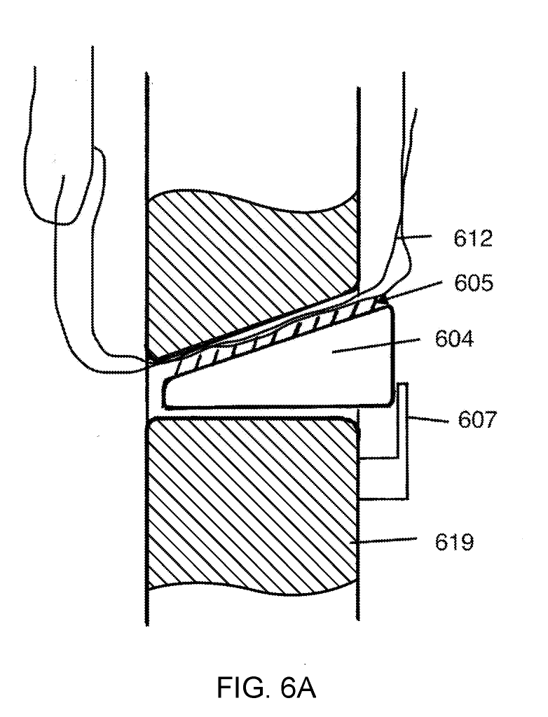

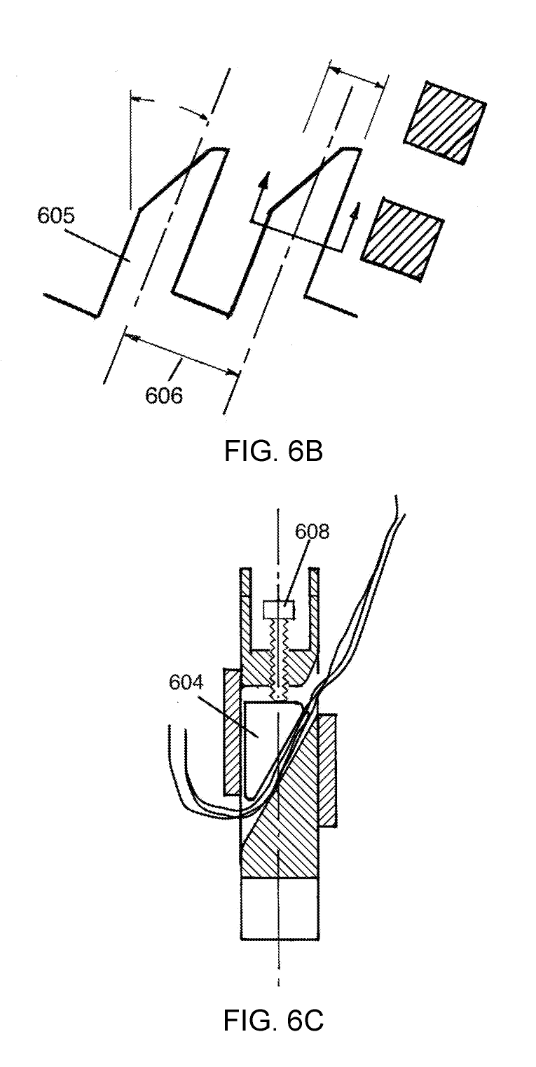

FIG. 6A shows a locking mechanism with mechanical tines 605 that engage the suture 612. The tines are small in dimension, with the spacing 606 between the tines 605 less than the diameter of the suture contemplated for use. As shown, the tines are tilted in one direction such that the suture can slide in the direction of tilt, but when the suture slides in the other direction, the tines dig into the suture, and cause pull on the wedge. As the suture pulls on the wedge 604, it is moved in a direction which closes the gap between the wedge 604 and the body 619, causing even tighter penetration of the tines 605 into the suture, and preventing sliding of the suture. This is an automatic locking mechanism. In FIG. 6A, a spring mechanism is also shown to hold the wedge lightly against the suture, to ensure the tines 605 catching of the suture as the suture slides in the direction of pull.

FIG. 6B demonstrates dimensions of the tines. The tines are small, as the sutures used may be less than ½ mm or 0.020 inch in diameter. The tines themselves maybe less than 0.25 mm, or 0.010 in. For typical suture diameters, the spacing interval (pitch) between the tines may be on the order of about 0.010 in. to about 0.020 in. At a distance of one-half the tine height, the tines may also be less than about 0.5 mm in width measuring the width of the tine at this half-height location above the plane of origin. The height of the tine is equal to or greater than the width of the tine, or may be equal to or greater than twice the width of the tine, measurement as described. The width of the tine may be stated as ½ the interval spacing of the tines. Preferentially, the tines are tilted, to be more active in one direction than another, and the tilt is greater than 5 degrees from the plane of origin of the tines. The tips of the tines may be pointed, to facilitate penetration of the tip of the tine between the fibers of the suture textile material. The tines may be used in a suture lock where the tines are positioned on a surface that is not a mobile wedge, but rather a substantially stationary part of the suture lock. In other cases, the tines are placed on a wedge surface, and are tilted in a direction aiming towards the wide end of the wedge. The wedges in general have a wedge angle less than about 45 degrees, and typically less than about 30 degrees. The wedge angle may be less than 25°.

FIG. 6C shows a preferred embodiment of a locking mechanism incorporated into a suture anchor. There is a wedge that is pushed by a threaded shaft 608, which by turning it, applies force and movement to the wedge, which in turn pinches the sutures passing through the suture locking mechanism. Anchors without pushing mechanisms are also considered. At the end of the anchor is the bone attachment mechanism, and at the other end of the anchor, is the end with the driver. Other mechanisms to advance the wedge against the suture are contemplated including an external removable pusher, contemplated to be part of the insertion tool. A mechanism to catch the wedge so it cannot slip back after pinching the suture is also considered. FIG. 6C can also represent an independent suture lock that is applied independently to a suture, as well as a suture anchor with an incorporated suture locking mechanism. Demonstrated in FIG. 6C is that the sutures enter and exit on the sides of the anchor body. Entry and exit may be on the same side of the body, or on opposite sides of the anchor body as shown. Also shown is the use of a band shown in cross-section, which is applied as a component of the body, used to restrain the wedge in proper position.

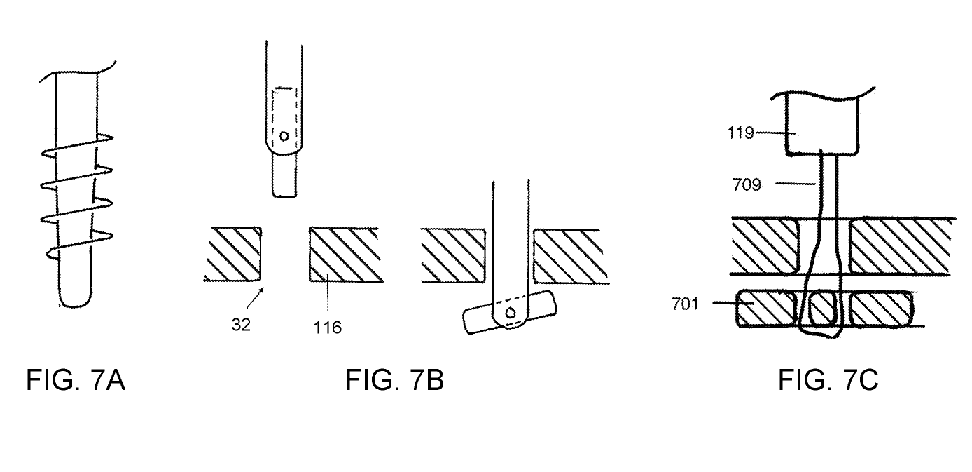

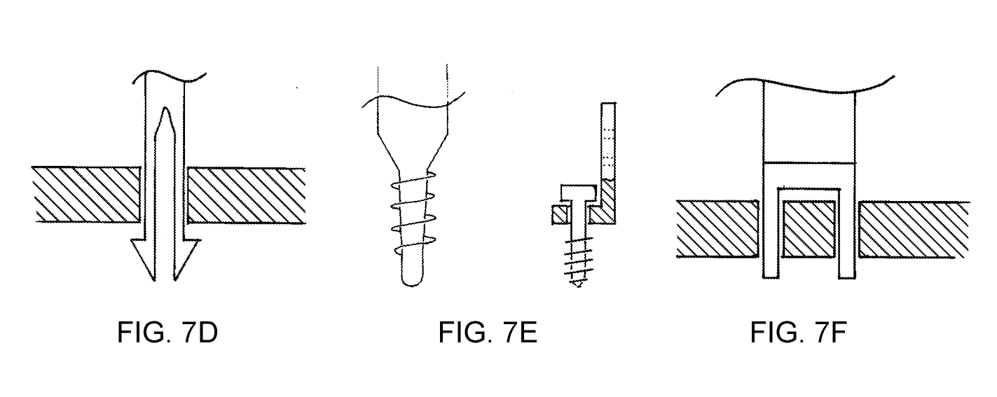

FIGS. 7A-7F show various bone attachment mechanisms/means, according to various embodiments.

FIG. 7A shows a thread which engages the cortical bone. Threaded BAM is the preferred embodiment, but the following bone attachment means are also contemplated and can be included in certain embodiments.

In FIG. 7A, there is a narrowing of the tip of the bone attachment mechanism, forming a nipple that is not threaded, and is lower diameter than the diameter of the thread and is in certain cases less than half the diameter of the thread. The specific sizing of the nipple is the same as that described for tap in FIG. 10.

FIG. 7B shows a toggle bolt mechanism 708 which after advancement through a hole in the bone, the mobile toggle element flips sideways, and prevents withdrawal from the hole after insertion through the hole.

FIG. 7C shows the bone attachment mechanism BAM consisting of a tether structure 709 attached to an elongate button 701. The button is oriented longitudinally, passed through the hole in the bone, flipped to lie transversely, such that traction on the body of the anchor will seat the button flat against the bone. This button restrains motion of the anchor away from the bone cortex, providing a structure for traction on the rotator cuff repair sutures, or sutures in other repaired structures. The button and tether serving as the bone attachment means allow the surgeon to make a small hole in the far cortex, minimally weakening the structure, and at the same time providing a stable attachment for the anchor body, to which the tissue repair sutures may be attached. In other examples of elongate button use, the sutures connect directly to the button, rather than having the button tether to an anchor body, which in turn is connected to the repaired tissues with sutures.

FIG. 7D shows a snap-lock mechanism 702 which relies on elasticity within the bone attachment mechanism BAM. As the bone attachment mechanism is pushed through the hole in the bone 720, sides of this snap-lock mechanism are forced toward the axis by the wedge shapes of the tips, making the distance between the sides 721 of the bone attachment mechanism low enough so that it may pass through the hole. Upon passing through the whole, the sides of the bone attachment mechanism snap out into their unstressed position, and withdrawal from the whole is prevented.

FIG. 7E shows another example of the bone attachment mechanism. The example of threaded BAM is shown in FIG. 7E. Other BAMs may be used as well. The transverse dimension of the bone attached mechanism is less than the transverse dimension of the anchor body, and it is in some cases less than 0.75 times the transverse dimension of the anchor body, and in other cases it is lesson 0.5 times the transverse dimension of the anchor body. Because the required dimension of the bone attachment mechanism is less than the required dimension of the body of the anchor, this is a particularly advantageous embodiment. This is opposite to the prior art, which typically relies on the surface area of the body and bone attachment, and bone attachment mechanism portion is the greatest transverse dimension of the anchor. According to the above embodiments, the anchor body sits within the central bone cavity, and there is plenty of space, and this opportunity is used to accommodate multiple sutures on one anchor, and one or more suture locking mechanisms in one SAC, which would not be possible with the small compact anchors used near the surface of the bone on the near side in prior art anchors, when attaching the rotator cuff tendon to the anatomic footprint insertion site.

FIG. 7E shows a suture anchor where the body of the anchor is free to rotate with respect to the BAM, according to an embodiment. The example of a threaded BAM is shown. Other BAMs including those shown in FIG. 7 may be used as well. In the case of this embodiment, it offers the special attribute of having the driver attached by a shaft to the BAM. The shaft between the driver and the BAM is part of the body of the anchor. In this case, the body of the anchor is in two parts, that are allowed to swivel with respect to one another. The part of the body that is allowed to swivel may have one or more holes 720, for example two holes or five holes, a multiplicity of Passage holes through the body. It may have locking mechanisms integrated into the body, or locking mechanisms attached to the suture that prevent sliding of the suture back through a hole in the body. According to various embodiments, BAM, anchor body, holes, and suture restraint mechanisms including locking mechanisms discussed herein are combined. The special attribute in this particular embodiment is that the driver may be twisted without twisting the part of the body having the holes, so that that the anchor can be advanced farther toward the far cortex without twisting the body having holes and sutures. This allows tightening of the tendon repair without loosening the sutures. The construct is optimized for the anatomic location of application, which is unique, and produces unique dimensions, and opportunities for multiple passage holes, which is not possible with suture anchors known in the art, and prior art does not allow tightening of the tendon repair in this fashion.

FIG. 7F shows yet another basic configuration for the BAM. In this case two or more elongate bone attachment elements 722 project out of the BAM, into a matching set of two or more holes 732 in the bone. There can be two or three elements 722. There can be four elements 722. Having multiple attachment projections into the bone provides rotational stability of the BAM, something much harder to provide with a single projecting element BAM. The rotational stability will provide resistance to torque applied to the anchor, in the process of twisting a screw to advance a wedge, for example. The options for elongate bone attachment elements 722 includes screws, for example.

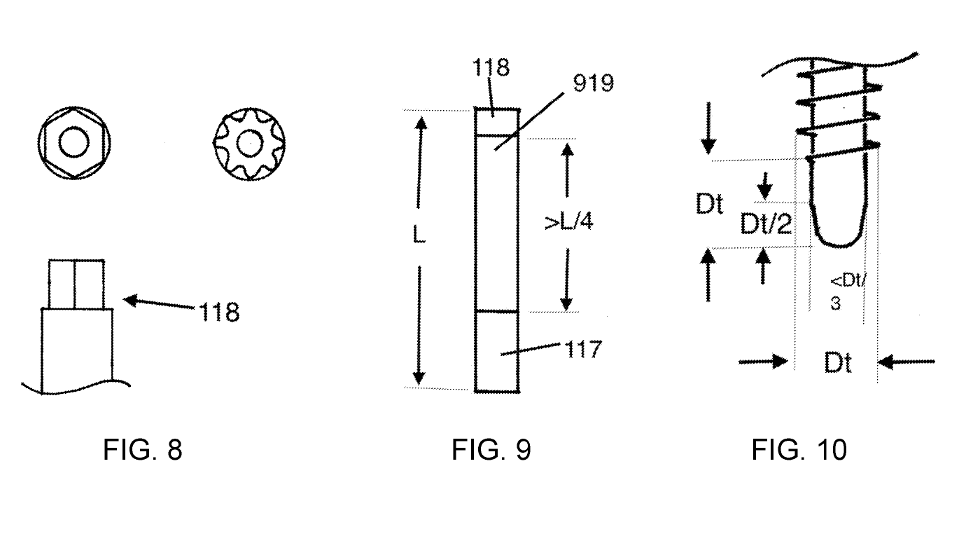

FIG. 8 shows the driver portion of the suture anchor, according to various embodiments. The driver portion includes a cross section that mates with the cross section profile on the insertion tool. Insertion tool that may be rotationally locked with respect to the driver, so that rotation of the insertion tool will cause rotation of the suture anchor. The cross-sectional geometry of the interface may be an irregular geometric shape or a regular geometric shape such as a square, hexagon. The cross-section may also represent the geometry of a Torx interface, or a splined interface. In the driver end of the anchor, there may be a hole, into which a threaded element can be advanced, for example to apply force to a locking wedge. In this case the driver for this threaded element would be coaxial with the insertion tool that engages the driver. Such a hole may also be used for passage of suture.

FIG. 9 shows the relationship in length between the BAM, body, and driver, according to various embodiments. Because the suture anchor is designed specifically to operate in the intraosseous space, more length of the body is possible, and it is also used to make the anchor effective, for example by making possible a series of holes along the body length. The body portion is defined as a portion of the suture anchor which is not the driver portion, and is not available for bone engagement in the far cortex. The body portion is equal to or greater than ¼ the length of the suture anchor. The body portion contains elements of the SAC including passage holes for the suture or sutures, and one or more locking mechanisms. There are embodiments of the SAC that do not require a long body 919, but there are others, including bodies with many holes and bodies with elongate locking mechanisms, where this length is required, and this length is not represented in the prior art.

FIG. 10 shows an instrument for use in surgical application of the anchor, according to various embodiments. This is a bone tap, for cutting threads in the bone. At the tip of the tap, there is a narrow portion without cutting threads. This tip is narrowed with respect to the diameter of the tap thread. At a distance of ½ half thread diameter Dt from the tip, the transverse diameter of the tip is equal to or less than one third of the tap thread diameter Dt. At a distance of one tap thread diameter from the tip, the diameter of the narrowed tip is less than one-half the thread diameter Dt of the tap. This tip enables the tap to find the bone entry hole 1020 at an oblique angle, as the tap blindly probes the far cortex.

In the case of metal suture anchors, the BAM may be self-drilling and tapping. In the case of plastic anchors, such as PEEK, the pre-drilling of the hole 132 is required. In some embodiments, use of both metal and/or plastic suture anchors is contemplated. The plastic anchors provide the specific advantage of being radiolucent, according to the preference of surgeons, and producing less imaging artifact on post-operative imaging.

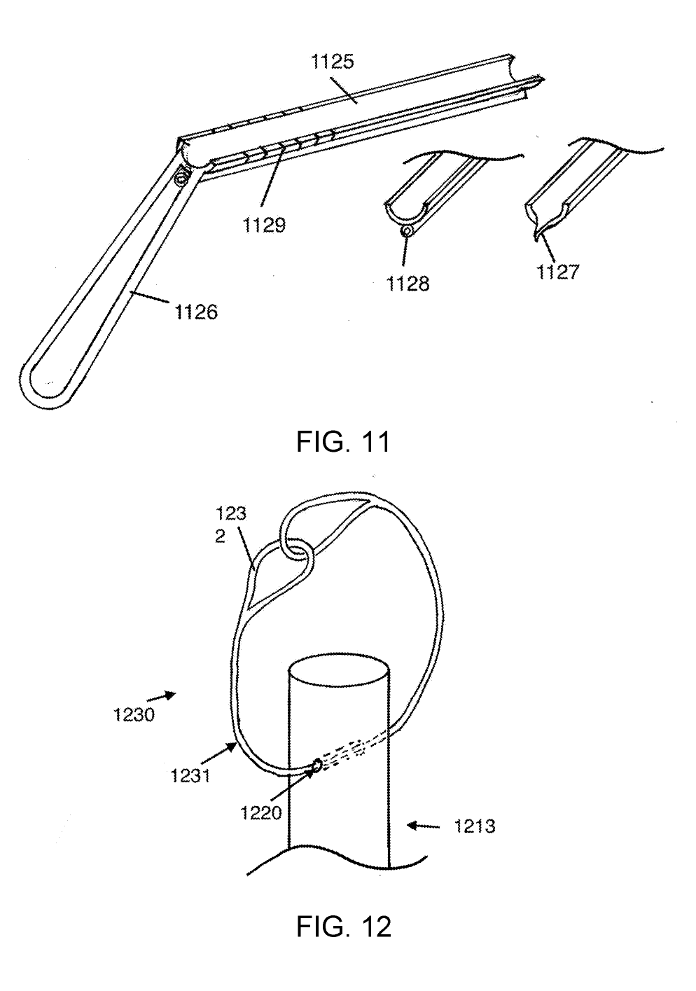

FIG. 11 shows a drill guide, according to various embodiments. The elements of the drill guide comprise a handle 1126 and a blade 1125. The blade portion may be shaped like a gutter, to allow exiting the guide sideways. The tip of the drill guide stabilizes the drill against the far cortex, and the drill guide is kept in position between drilling, tapping, and insertion of the anchor. In this way, the hole 132 on the far cortex, that cannot be visualized directly, is not lost, and time is not lost re-finding this hole by probing. The drill guide may have a spike 1127 at the tip, that digs into the endosteal surface of the far cortex 116, and keeps position of the drill guide. Alternatively the drill guide may have an additional channel 1128 for passage of a small Kirschner wire (also referred to as K-wire) down the length of the drill guide and into the far cortex 16, holding the drill guide from movement between the operations of hole drilling, tapping, and anchor insertion. The gutter shaped drill guide is open on one side, on the side away from the handle, also so that the anchor which typically has a wider body than BAM, may be advanced down the drill guide without being captured and blocked by the radius of the drill guide. An additional feature of the drill guide in FIG. 11 is the length graduations 1129 marked on the blade of the drill guide. These graduations are used to inform the surgeon the length of anchor required for use. When the drill guide is placed against the far cortex, it is essentially a depth gauge, showing the distance between the greater tuberosity, and the far cortex. This is the maximum distance that can be used by the anchor, and typically a fraction of this distance, such as 0.7× this distance or 0.5× this distance is used by the length of the suture anchor body and driver. The graduations of the drill guide do not accommodate or account for the length of the BAM that projects through the far cortex and out the other side.

FIG. 12 shows a passing loop 1230 that may be positioned in the holes 1220 or suture channels of the anchor, according to an embodiment. In some embodiments, the SAC is supplied with one or more passing loops already in place, so when the surgeon takes the anchor out of the package, these passing loops are already conveniently in place. The passing loops are used to drag the rotator cuff sutures through the suture holes 1220 in the anchor 1213, after placing the anchor. The passing loops also may be used prior to insertion of the anchor into the central cavity 14, in the case where the anchor does not need to be rotated for insertion such as with a threaded separate bone attachment mechanism as in FIG. 7E. In FIG. 12, a special passing loop is shown, where throughout greater than 80% of the length of the loop, the passing Loop has only one tether cord 1231. In this case the suture is drawn into the hollow braid, exposing only a loop 1232 at the end, where the suture has not been drawn into its own hollow braid. With the passing loop disclosed, the passing loop can be passed multiple time around through the same hole in the same direction.

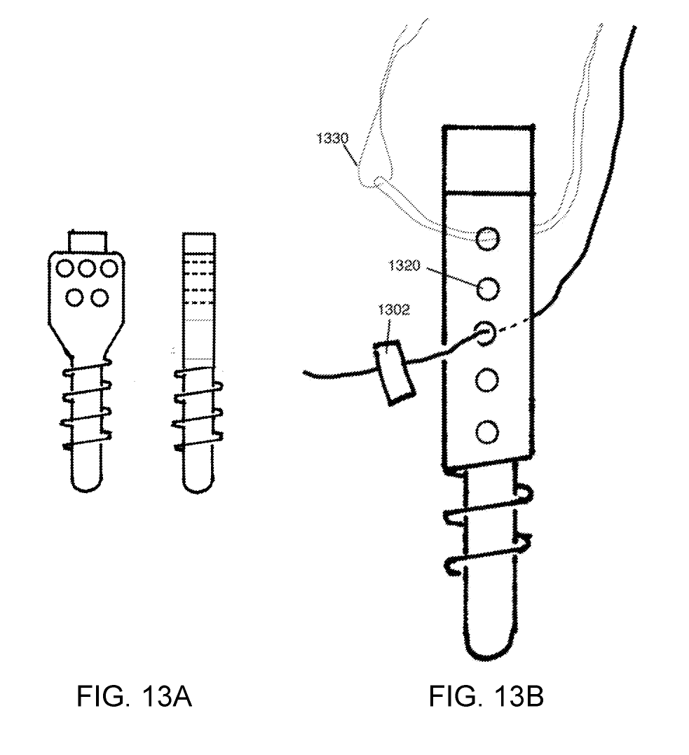

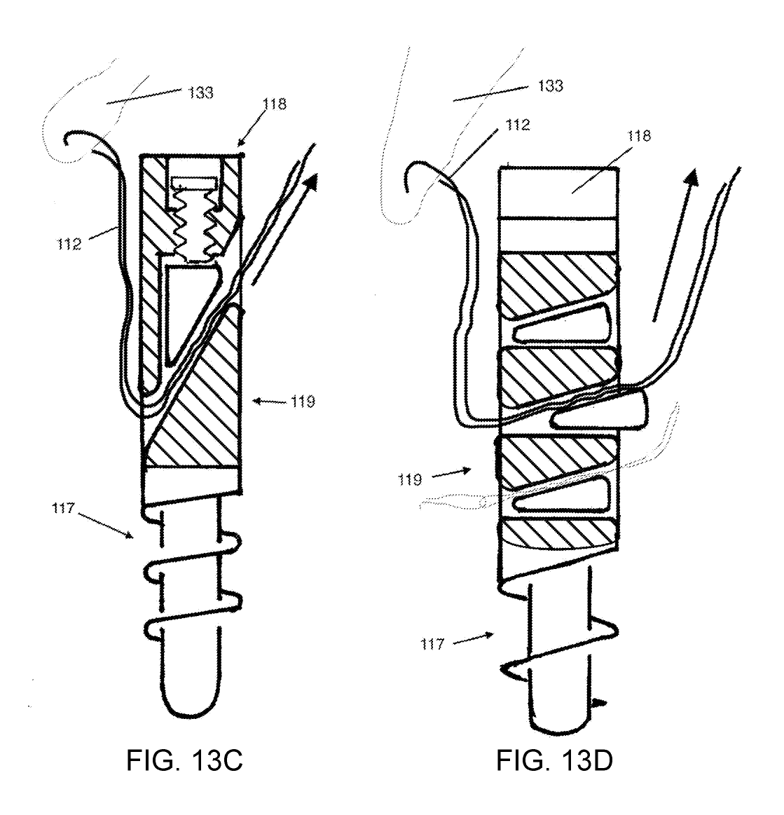

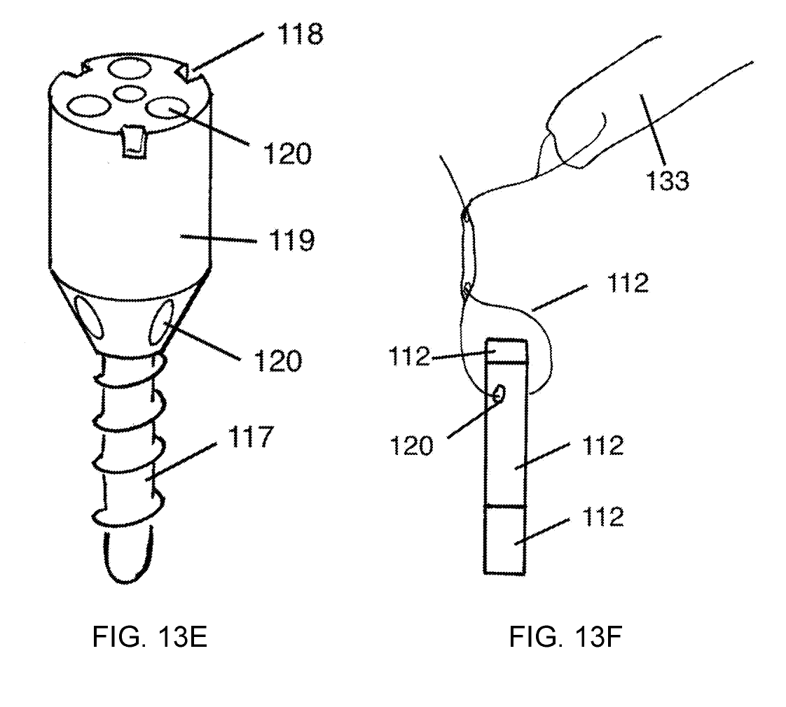

FIGS. 13A-13F show the suture anchor according to various embodiments.

FIG. 13A shows an anchor where there are transverse holes in the anchor body portion, according to various embodiments. In such embodiments, the anchor body portion can be wider than the bone attachment portion by a factor of greater than about 1.5. Multiple holes are providing suture passage through the anchor. The holes shown in FIG. 13A may or not may or may not include suture locking mechanisms. The holes are clustered so that some of the holes are substantially side-by-side. This reduces the required length of the body, to offer the same number of holes. There may be two holes, 3 holes, 4 holes, or greater than 4 holes. At least two of the holes are positioned where the distance of the hole from the tip of the BAM 1301 is less than one hole diameter different between the two holes.

FIG. 13B shows a more elongate SAC configuration, where the holes through the body are arranged sequentially along the length of the body, according to various embodiments. A suture is shown passed through one of the holes in the body for demonstration. A suture lock 1302 is clamped onto the suture, preventing the suture from being drawn back through the hole in the body. The same means for restraining the sutures is repeated for all other sutures passing through holes in the body. The holes through the body may be in different directions, they may be transverse to the axis, and different radial directions with respect to the axis, and they may be oblique to the axis. The holes may have rounded edges, to prevent cutting of the suture as it is drawn through the hole to tighten the repair. The SAC may be provided with passing loops 1330 through the holes 320, to facilitate passage of sutures through the holes intraoperatively.

FIG. 13C shows a suture anchor with a locking mechanism incorporated into the body of the anchor, according to various embodiments. The locking mechanism is a single wedge, with the wedge oriented longitudinally with respect to the axis of the suture anchor. The driver portions and BAM portions of this anchor have several options, and these are discussed earlier in this specification.

FIG. 13D shows a suture anchor where multiple substantially transverse holes in the body each contain a wedge locking mechanism. The direction of motion of the wedge in association with locking is transverse to the axis of the suture anchor. The transfers holes through the anchor, that include the locking mechanism, have rounded edges, too offer a smooth gliding surface to the suture that is drawn through these holes. This anchor may be supplied with a passing loop in each of the transfers holes when it is provided to the surgeon. At the end of the surgery, the passing loop may be removed and discarded, or incorporated into the tendon repair.

FIG. 13E shows a suture anchor where the passage holes in the body of the anchor are longitudinal, substantially parallel to the axis of the suture anchor, according to an embodiment. This embodiment is different from the others in that there are multiple holes in the body of the anchor, oriented longitudinally. These holes may contain locking mechanisms, such as the wedge locking mechanism, with tines, or other locking mechanisms. In this example, the transverse dimension of the body is more than 1.5 times the transverse dimension of the bone attachment mechanism BAM.

FIG. 13F illustrates a suture employed in the embodiments. However, suture employed in various embodiment may be any kind of suture, including braided sutures, monofilament sutures, flat braid sutures, and variable denier sutures. In some embodiments, braided sutures and variable denier sutures are preferentially employed that include braided elements. Variable denier sutures are with less suture material at the ends, making them narrower at the ends of the suture then in a central portion of the suture. This allows the end of the suture to be doubled over, and still retain a cross-section of the double/portion that is equal to or less than the cross section of the full thickness central portion. This allows the variable denier suture to be doubled over at the end, and drawn through a hole, such as the hole in the suture anchor, and after the doubled over portion is pulled through, the thicker central portion is pulled in, more nearly filling up the entire passage hole through the anchor. This offers considerable advantage and fully utilizing the suture carrying capacity of the anchor, and also reduces the amount of closure required by the locking mechanisms before clamping and locking of the suture is achieved. If a double width channel is required to pass a loop of suture, and then after the suture is pulled through the channel, it must be closed down to single width to lock the suture, and a much greater excursion of the locking mechanism is required. Therefore it is of substantial advantage to use variable denier sutures in the automatic locking mechanisms. Another application of variable denier sutures is to draw the end portion through the central channel of suture itself, creating a “Chinese finger trap” lock so that it is a self-locking suture. In various embodiments, self-locking sutures may be used to hold the repaired tendon and restrain the suture in the same way as a knot.

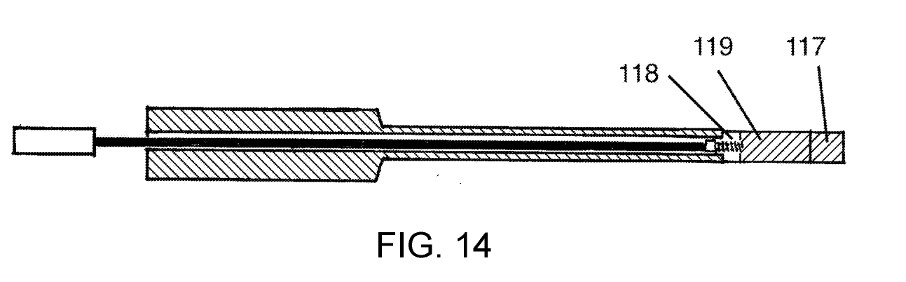

FIG. 14 illustrates an insertion tool.

The insertion tool may be part of the SAC construct. It has a handle, a shaft, a driver engagement portion. The driver engagement portion interface mates with the driver and may allow torque and application of twist to the suture anchor. The shaft may be tubular, accommodating either a traction tether to hold the anchor onto the tool, or to receive an elongate driver to apply twist to the suture locking mechanism of the anchor.

FIGS. 15A-15C have been described above.

While the foregoing is directed to embodiments of the present invention, other and further embodiments may be devised without departing from the basic scope thereof, and the scope thereof is determined by the claims that follow.

What is claimed is:

1. A suture anchor, comprising:
a bone attachment portion configured for engagement in cortical bone; and
a body portion that is coupled to the bone attachment portion and includes a plurality of holes for passage of sutures.

2. The suture anchor of claim 1, wherein the body portion has a length that is at least one-fourth of an entire length of the suture anchor.

3. The suture anchor of claim 1, wherein the bone attachment portion has a length that is less than one-fourth of an entire length of the suture anchor.

4. The suture anchor of claim 1, wherein each of the bone attachment portion and the body portion has a generally cylindrical shape.

5. The suture anchor of claim 4, wherein a diameter of the bone attachment portion is less than a diameter of the body portion.

6. The suture anchor of claim 1, further comprising:
a suture locking mechanism configured to secure the sutures passing through the holes to the body portion.

7. The suture anchor of claim 6, wherein the suture locking mechanism includes a movable wedge that is pressed against the body portion to secure one of the sutures passing through one of the holes to the body portion.

8. The suture anchor of claim 7, wherein the movable wedge has a plurality of tines formed on a surface thereof that presses the suture against the body portion.

9. The suture anchor of claim 8, wherein the tines are spaced from each other at an interval that is less than the diameter of the suture.

10. The suture anchor of claim 8, wherein the tines are tilted in a first direction that is opposite a second direction in which the movable wedge is moved to press the suture against the body portion.

11. The suture anchor of claim 6, wherein the suture locking mechanism includes multiple wedges that are mechanically linked to move together to press the sutures passing through the holes against the body portion to secure the sutures to the body portion.

12. The suture anchor of claim 1, further comprising:
a suture locking mechanism including a surface locking mechanism that is attached to the sutures passing through the holes and pressed against the body portion.
